(19) United States
(12) Patent Application Publication
(10) Pub. No.: US 2011/0186428 A1
(43) Pub. Date: Aug. 4, 2011

(54) ELECTRODE ARRANGEMENTS FOR BIOSENSORS

(75) Inventors: Terry Beaty, Indianapolis, IN (US); Henning Groll, Tucson, AZ (US); Harvey Buck, Indianapolis, IN (US); Eric Diebold, Fishers, IN (US); Abner Joseph, Camel, IN (US); Randy Riggles, Indianapolis, IN (US)

(73) Assignee: Roche Diagnostics Operations, Inc., Indianapolis, IN (US)

(21) Appl. No.: 12/696,316

(22) Filed: Jan. 29, 2010

(51) Int. Cl. G01N 33/50

(52) U.S. Cl. 204/403.01

(57) ABSTRACT

The present invention relates to a biosensor. The biosensor includes a support substrate, electrodes positioned on the support substrate, a spacer substrate positioned on the support substrate, and a cover positioned on the spacer substrate. The cover cooperates with the support substrate to define a capillary channel. The electrodes include at least one working electrode defining a working electrode area in the capillary channel. The working electrode is configured to minimize variation in the working electrode area in the capillary channel due to variations in the spacer substrate placement relative to the working electrode.

### BACKGROUND

Electrochemical biosensors are known. They have been used to determine the concentration of various analytes from biological samples, particularly from blood. Electrochemical biosensors are described in U.S. Pat. Nos. 5,413,690; 5,762,770; 5,798,031; 5,997,8171; 7,073,246; 7,195,705; and 7,473,398 and U.S. Patent Application Publication No. 2005/0016844, the disclosure of each of which is expressly incorporated herein by reference.

For example, as the number of patients suffering from diabetes and similar medical conditions increases, self-monitoring of blood glucose wherein the patient monitors his or her blood glucose levels has become a common practice. The purpose of monitoring the blood glucose level is to determine the concentration level and then to take corrective action, based upon whether the level is too high or too low, to bring the level back within a normal range. The failure to take corrective action can have serious medical implications. Glucose monitoring is a fact of everyday life for diabetic individuals. Failure to test blood glucose levels properly and on a regular basis can result in serious diabetes-related complications, including cardiovascular disease, kidney disease, nerve damage and blindness.

A number of biosensors employ electrochemical analysis to determine the blood glucose level by measuring a current related to an analyte concentration. Such biosensors may employ a capillary channel with an electrode substrate providing a working electrode area in the capillary channel. The current response of the electrochemical cell is directly proportional to the working electrode area. However, variations in the working electrode area are created during the manufacture and assembly of the components of the biosensor that define the capillary channel. Variations in the working electrode area in the capillary channel from one biosensor to another are undesirable since the variation in electrode area introduces imprecision in the measured analyte concentration. Therefore, biosensor arrangements which minimize variations in the working electrode area in the manufacture of the biosensor are desirable.

### SUMMARY

The present invention relates to a biosensor. The biosensor includes a support substrate, electrodes positioned on the support substrate, a spacer substrate positioned on the support substrate, and a cover positioned on the spacer substrate. The cover cooperates with the support substrate to define a capillary channel. The electrodes include at least one working electrode defining a working electrode area in the capillary channel. The working electrode is configured to minimize variation of the effective working electrode area in the capillary channel due to variations in the spacer substrate placement relative to the working electrode while also maximizing the effective working electrode area within the capillary channel.

According to one aspect, a biosensor comprises a support substrate extending between opposite first and second ends and opposite first and second edges; a spacer substrate positioned on the support substrate that includes an inner edge extending along the support substrate between the first and second ends and the first and second edges; a cover cooperating with the spacer substrate so that the inner edge of the spacer substrate defines a boundary of a capillary channel; and at least one working electrode in the capillary channel. The working electrode includes a width and a main body portion extending along a length transversely to the width between opposite ends of the main body portion. The main body portion includes at least two working electrode portions positioned along the length of the main body portion in the capillary channel with the at least two working electrode portions connected by at least one connecting portion. The working electrode further includes at least one connective neck extending from at least one of the opposite ends of the main body portion and across the inner edge of the spacer substrate. The two working electrode portions each define a minimum or least width that is greater than a maximum or greatest width of the connective neck, and the connecting portion defines a maximum or greatest width that is less than a minimum or least width of the connective neck.

In one refinement of the aspect, the capillary channel includes an inlet at the first end of the support substrate and the main body portion of the working electrode is located entirely within the capillary channel.

In a further refinement of the aspect, the working electrode includes a second neck extending from the other of the opposite ends of the main body portion in the capillary channel, the second neck extending across the inner edge of the spacer substrate.

In a further refinement of the aspect, the working electrode includes first and second connecting portions extending between and connecting the at least two working electrode portions to one another in the capillary channel. The first and second connecting portions each include a maximum or greatest width that is less than the minimum or least width of the connective neck, and the first and second connecting portions are separated from one another by a non-conductive space between the connecting portions and working electrode portions.

In another refinement of the aspect, the at least one connecting portion of the working electrode includes a plurality of rows of connecting portions extending between the at least two working electrode portions of the working electrode. Adjacent pairs of the rows of connecting portions are separated from one another by a non-conductive space, and each row of the connecting portions includes a maximum or greatest width that is less than the minimum or least width of the at least one connective neck.

In another refinement of the aspect, the at least two working electrode portions of the main body portion of the working electrode includes a plurality of working electrode portions spaced along the plurality of rows of connecting portions to form a grid-shaped pattern for the main body portion of the working electrode.

According to another aspect, a biosensor comprises a support substrate extending between opposite first and second ends and opposite first and second edges; a spacer substrate positioned on the support substrate that includes an inner edge extending along the support substrate with the inner edge being located between the first and second ends and the first and second edges of the support substrate; a cover cooperating with the spacer substrate so that the inner edge of the spacer substrate defines a boundary of a capillary channel; and at least one working electrode. The at least one working electrode includes a main body portion defining a width and a length transverse to the width between opposite ends of the main body portion. The length and width are sized so that the main body portion is located in the capillary channel. The working electrode further includes first and second connective necks each extending from a respective one of the opposite ends of the main body portion and across the inner edge of the spacer substrate. The main body portion defines a minimum or least width that is greater than a maximum or greatest width of each of the first and second necks. Each of the first and second connective necks extends from the main body portion to an electrode lead on the support substrate so that each of the first and second connective necks provides an electrical connection with the working electrode.

In one refinement of the aspect, the main body portion of the working electrode includes a maximum width at a center of the main body portion and tapers in width from the center toward each of the first and second connective necks.

In another refinement of the aspect, the first connective neck extends to an electrode lead that extends along the support substrate to an electrode contact, and the second connective neck extends to an electrode looping portion located outside the capillary channel. The electrode looping portion joins the second connective neck to the electrode lead so that the working electrode forms a continuous loop located within and outside the capillary channel.

According to another aspect, a biosensor comprises a support substrate extending between opposite first and second ends and opposite first and second edges; a spacer substrate positioned on the support substrate that includes an inner edge extending along the support substrate, the inner edge extending from the first edge to the second edge adjacent the first end of the support substrate; a cover cooperating with the spacer substrate so that the inner edge of the spacer substrate defines a boundary of a capillary channel; and at least one working electrode in the capillary channel. The working electrode includes a main body portion with a length that extends toward the first and second edges within the capillary channel. The working electrode further includes a connective neck extending from an end of the main body portion toward the second end of the support substrate. The inner edge is spaced from the main body portion and extends across the connective neck where the connective neck is oriented to extend toward the second end of the support substrate.

In one refinement of the aspect, the main body portion of the working electrode is located entirely within the capillary channel.

In another refinement of the aspect, the working electrode includes first and second connective necks extending from opposite ends of the main body portion toward the second end of the support substrate and the inner edge extends across each of the first and second connective necks where the first and second connective necks are oriented toward the second end of the support substrate.

In another refinement of the aspect, the main body portion includes a minimum or least width along a substantial portion of the length and the connective neck includes a maximum or greatest width as measured in a direction toward the first and second edges of the support substrate, the minimum width of the main body portion being greater than the maximum width of the connective neck.

According to another aspect, a method for manufacturing a biosensor comprises: providing a support substrate; forming at least one working electrode on the support substrate, the working electrode including a main body portion and at least one connective neck extending from an end of the main body portion, wherein a width of the at least one connective neck is greater than a minimum or least width of part of the main body portion of the working electrode; and positioning a spacer substrate on the support substrate, the spacer substrate including an inner edge that defines a boundary of a capillary channel, the inner edge extending across the at least one connective neck of the working electrode so that the part of the main body portion defining the minimum width is located entirely within the capillary channel.

According to another aspect, a method for manufacturing a biosensor comprises: providing a support substrate; forming at least one working electrode on the support substrate, the working electrode including a main body portion defining a substantially constant width along a substantial portion of a length of the main body portion, the working electrode including a central portion projecting outwardly from the width; and positioning a spacer substrate on the support substrate so that opposite portions of an inner edge of the spacer substrate extend across opposite lateral portions of the main body portion and the central portion of the working electrode is positioned entirely within a capillary channel defined by portions of the inner edge, wherein the central portion occupies less than half of the length of the main body portion between the portions of the inner edge.

Further aspects, embodiments, forms, features, benefits, objects, and advantages shall become apparent from the detailed description and figures provided herewith.

### BRIEF DESCRIPTION OF THE FIGURES

FIG. 1 is a perspective view of one embodiment biosensor.

FIG. 2 is a plan view with portions shown in partial phantom of the biosensor of FIG. 1.

FIG. 3 is cross-section view of a portion of the biosensor of FIG. 1 along view line 3-3.

FIG. 4 is a plan view of a portion of the biosensor of FIG. 1 showing a sample revising chamber and electrode arrangement.

FIG. 5 is a plan view of another embodiment capillary channel and electrode arrangement.

FIG. 6 is a plan view of another embodiment capillary channel and electrode arrangement.

FIG. 7 is a plan view of another embodiment capillary channel and electrode arrangement.

FIG. 8 is a plan view of another embodiment capillary channel and electrode arrangement.

FIG. 9 is a plan view of another embodiment capillary channel and electrode arrangement.

FIG. 10 is a plan view of another embodiment capillary channel and electrode arrangement.

FIG. 11 is a plan view of another embodiment capillary channel and electrode arrangement.

FIG. 12 is a plan view of another embodiment capillary channel and electrode arrangement.

FIG. 13 is a plan view of another embodiment capillary channel and electrode arrangement.

FIG. 14 is a plan view of another embodiment capillary channel and electrode arrangement.

FIG. 15 is a plan view of another embodiment capillary channel and electrode arrangement.

FIG. 16 is a plan view of another embodiment capillary channel and electrode arrangement.

FIG. 17 is a plan view of another embodiment capillary channel and electrode arrangement.

### DETAILED DESCRIPTION OF THE ILLUSTRATED EMBODIMENTS

For purposes of promoting an understanding of the principles of the invention, reference will now be made to the embodiments illustrated in the drawings and specific language will be used to describe the same. It will nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates.

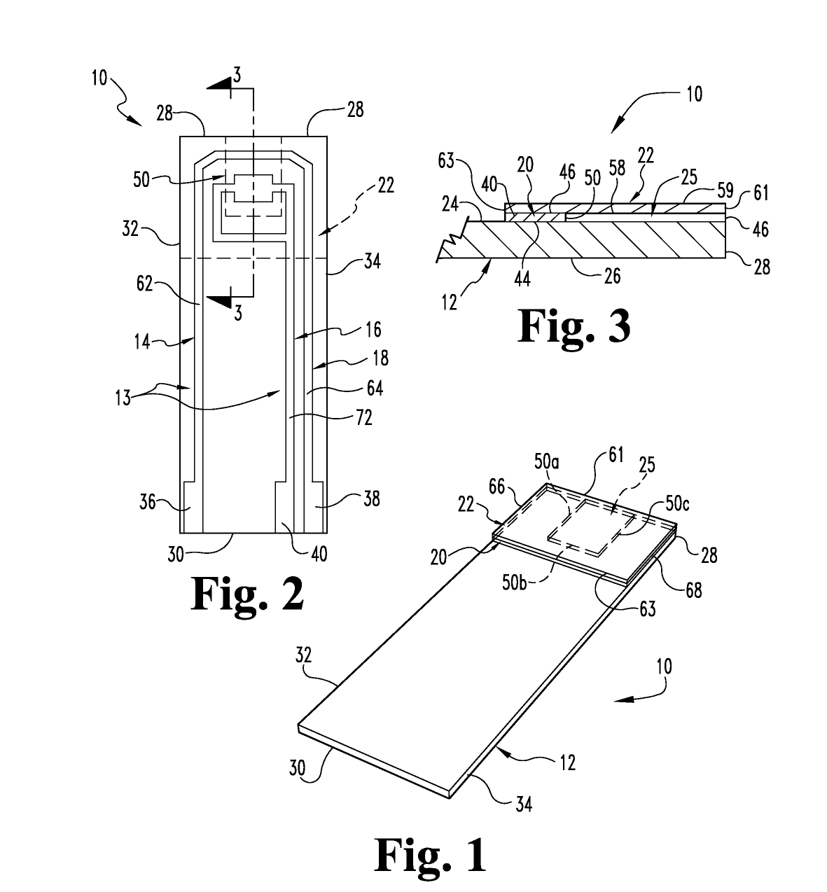

The present invention relates to a biosensor and a method for manufacturing a biosensor that reduces sensitivity of the effective working electrode area to manufacturing variations. The present invention improves precision and accuracy of current measurements in electrochemical analysis of an analyte positioned in a capillary channel of the biosensor in contact with the effective working electrode area. The biosensor and method for manufacturing are relatively low in cost since the advantages are achieved without necessarily requiring significant additional steps or materials in the manufacturing process, such would be involved in screen printing of insulating overlays to define the working electrode area. Aspects of the invention are presented in FIGS. 1-17, which are not drawn to scale and wherein like components in the several views are numbered alike.

FIGS. 1-3 illustrate an aspect of the invention in the form of a biosensor 10 having an electrode-support substrate 12, an electrical conductor 13 positioned on the support substrate 12 that defines electrodes 14, 16, 18, a spacer substrate 20 positioned on support substrate 12, and a cover 22 positioned on the spacer substrate 20. Spacer substrate 20 defines a capillary channel 25 along support substrate 12. Electrodes 14, 16, 18 include at least one working electrode that defines an effective working electrode area in capillary channel. The effective working electrode area is the area of the working electrode that contacts a fluid sample in capillary channel 25 when the capillary channel 25 includes sufficient volume of the fluid sample to initiate measurement sequence.

Biosensor 10 is shown as rectangular in shape, it being understood, however, that biosensor 10 can be provided in any one of a number of shapes in accordance with principles of this disclosure. Furthermore, biosensor 10 can be any one of a substantial quantity of biosensors produced from rolls of material, sheets of material, or other material stock in accordance with the principles of this disclosure. In one embodiment, the selection of materials for the construction of biosensor 10 includes a stock sufficiently flexible for roll processing, but still rigid enough to give a useful stiffness to finished biosensor 10. The biosensor arrangement and method for manufacturing the biosensor described herein minimizes variations in effective working electrode area from one biosensor to the next, improving precision and accuracy of current readings measured by the working electrode during electrochemical analysis of a fluid sample.

Variation in effective working electrode area can be caused by imprecision in forming the working electrode, or at least the portion of the working electrode exposed within the capillary channel. However, the variation problem attempted to be solved by the present invention is caused by imprecision in forming the capillary channel itself where the effective working area is exposed. For a biosensor which utilizes a spacer layer to define the capillary channel, imprecision may lie in the inner edge or edges formed in the spacer layer to define the capillary channel. This affects effective working electrode area where the working electrode extends across that inner edge, wherein deviation of the inner edge of the spacer at that location directly increases or decreases the exposed portion of the working electrode within the capillary channel, thereby increasing or decreasing the effective working electrode area. Thus, the present invention relates to working electrode configurations designed to minimize the overall impact of imprecision of the inner edge on the total working electrode area exposed in the capillary channel.

The electrode-support substrate 12 is shown in FIGS. 2 and 3. Support substrate 12 includes a first surface 24 facing the spacer substrate 20 and a second surface 26 opposite first surface 24. In addition, support substrate 12 has opposite first and second ends 28, 30 and opposite edges 32, 34 extending between the first and second ends 28, 30. While ends 28, 30 and edges 32, 34 of support substrate 12 are illustrated to form a generally rectangular shape, it should be understood that the ends and edges of support substrate 12 may form any one of a variety of shapes and sizes in accordance with the principles of this disclosure. In one specific embodiment, support substrate 12 can be formed of a flexible polymer, including, for example, a polyester or polyimide, such as polyethylene naphthalate (PEN). Other suitable materials for support substrate 12 as would occur to one of ordinary skill in the art are also contemplated.

Electrodes 14, 16, 18 are formed from conductor 13 provided on first surface 24 of support substrate 12. Non-limiting examples of material suitable for electrical conductor 13 include aluminum, carbon (such as graphite), cobalt, copper, gallium, gold, indium, iridium, iron, lead, magnesium, mercury (as an amalgam), nickel, niobium, osmium, palladium, platinum, rhenium, rhodium, selenium, silicon (such as highly doped polycrystalline silicon), silver, tantalum, tin, titanium, tungsten, uranium, vanadium, zinc, zirconium, mixtures thereof, and alloys, oxides, or metallic compounds of these elements. In one specific embodiment, electrodes 14, 16, 18 are isolated from the rest of the electrical conductor 13 by laser ablation or laser scribing, and electrodes 14, 16, 18 are created by removing the electrical conductor 13 from an area extending around the electrodes either broadly, such as by broad field ablation, or minimally, such as by line scribing. Other embodiments contemplate other techniques for forming electrodes 14, 16, 18 as would occur to those of ordinary skill in the art, such as lamination, screen-printing, or photolithography.

Electrodes 14 and 18 define reference or counter electrode 60 and electrode 16 defines working electrode 70, at least a portion of each of which are located in capillary channel 25. Leads 62, 64 extend away from the counter electrode 60, and lead 72 extends away from working electrode 70. Leads 62, 64, 72 extend from the electrodes 60, 70 to contacts 36, 38, 40, respectively, at the second end 30 of the electrode-support substrate 12. Contacts 36, 38, 40 provide an electrical connection with a meter (not shown) or other device when biosensor 10 is positioned therein. It is contemplated that the leads 62, 64, 72 extending from the electrodes 60, 70 can be formed to have any suitable length and extend to any suitable location on the electrode-support substrate 12. It is further contemplated that the configuration of the electrodes, the number of electrodes, as well as the spacing between the electrodes may vary in accordance with this disclosure and that more than two electrodes may be formed as illustrated and discussed further herein.

Spacer substrate 20 of biosensor 10 includes a first member 40 extending between the edges 32, 34 of the electrode-support substrate 12. It is contemplated that spacer substrate 20 may be comprised of a single member or a plurality of members. First member 40 includes an inner edge 50 facing the capillary channel 25 and defining a boundary of capillary channel 25. In the illustrated embodiment of FIGS. 1-3, the inner edge 50 includes multiple portions 50a, 50b, 50c located between ends 28, 30 and edges 32, 34. Edge portions 50a, 50b, 50c extend along at least three sides of the capillary channel 25 in a generally U-shaped pattern to define the boundary of capillary channel 25 having a sample inlet 46 at an end 28 of the biosensor. The inlet 46 may also be provided at one of the edges 32, 34 as desired (not shown). Other embodiments contemplate an inner edge 50 that is linear, such as shown in FIGS. 15-17. Still other embodiments contemplate an inner edge 50 that forms hemi-ovular, semi-circular, or other shaped capillary channels, and the one or more of the portions of inner edge 50 may include linear or non-linear edges along all or part of its length.

When spacer substrate 20 is coupled to support substrate 12, electrodes 60 and 70 are positioned to lie within the capillary channel 25 formed by spacer substrate 20 between support substrate 12 and cover 22. Any variation in the width of the capillary channel 25 defined by inner edge 50 introduces variation in the effective area of working electrode 70 that is located in capillary channel 25, resulting in imprecision of the current measured related to an analyte concentration. Biosensor 10 is arranged to maximize the effective area of working electrode 70 certain to be exposed when spacer substrate 20 is positioned on support substrate 12 relative to the effective area of working electrode 70 that may be unintentionally exposed or covered by spacer substrate 20.

Spacer substrate 20 is formed from an insulative material, such as, for example, a flexible polymer including an adhesive coated polyethylene terephthalate (PET)-polyester. A non-limiting example of a suitable material includes a white PET film, both sides of which are coated with a pressure-sensitive adhesive. It is contemplated that spacer substrate 20 may be constructed of a variety of materials and includes an inner surface 44 that may be coupled to support substrate 12 and an outer surface 46 coupled to the cover substrate 22 using any one or combination of a wide variety of commercially available adhesives. Additionally, when surface 24 of support substrate 12 is exposed and not covered by electrical conductor 13, spacer substrate 20 may be coupled to support substrate 12 by welding, such as heat or ultrasonic welding. It is also contemplated that first surface 24 of support substrate 12 may be printed with, for example, product labeling or instructions (not shown) for use of biosensor 10.

Cover substrate 22 is coupled to upper surface 46 of spacer substrate 20. Cover substrate 22 includes an inner surface 58 facing spacer substrate 20 and an outer surface 59. In addition, cover substrate 22 includes opposite first and second ends 61, 63 and edges 66, 68 extending between the first and second ends 61, 63. When biosensor 10 is assembled, cover 22 cooperates with the spacer support substrate 20 and the electrode-support substrate 12 to define a sample receiving chamber or capillary channel 25. Cover substrate 22 is generally rectangular in shape; it is appreciated, however, that the cover substrate 22 may be formed in one of a variety of shapes and sizes in accordance with the principles of this disclosure. Cover substrate 22 may be formed from a flexible polymer and preferably from a polymer such as a polyester or polyimide. A non-limiting example of a suitable polymer is a hydrophilic polyester film.

Referring now to FIG. 3, capillary channel 25 includes a sample inlet 46 between cover 22 and support substrate 12 adjacent to ends 61 and 28. As shown in FIGS. 1 and 2, capillary channel 25 is located between edges 32, 66 and edges 34, 68 respectively. Capillary channel 25 may also include one or more holes through cover 22 or additional channels extending to edges 32, 66 and/or edges 34, 68 that serve as air outlets. Capillary channel 25 is also defined by inner edge 50 of first member 40 of the spacer substrate 20. Therefore, when biosensor 10 is assembled, capillary channel 25 extends across at least a portion of counter and working electrodes 60, 70.

It is further contemplated that electrochemical reagents can be positioned on counter and working electrodes 60, 70. The reagents provide electrochemical probes for specific analytes. The choice of specific reagents depend on the specific analyte or analytes to be measured, and are well known to those of ordinary skill in the art. An example of a reagent that may be used in biosensor 10 is a reagent for measuring glucose from a whole blood sample.

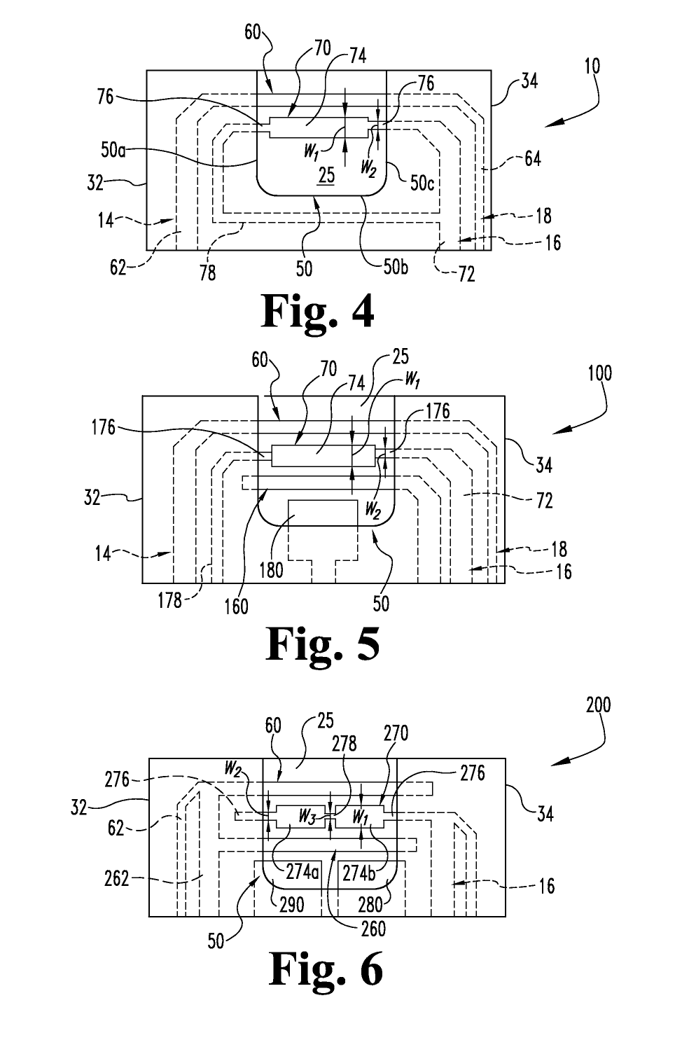

One arrangement of counter electrode 60 and working electrode 70 in capillary channel 25 is further shown in FIG. 4. Working electrode 70 includes a main body portion 74 having length between opposite ends, and a minimum or least width W1 transverse to and along a substantial portion of its length. The length and width are sized so that main body portion 74 is located entirely in capillary channel 25. Connective necks 76 extend from opposite ends of main body portion and across inner edge 50. Connective necks 76 each have a maximum or greatest width W2 that is substantially less than minimum width W1. Connective necks 76 include a length sized so that portions 50a, 50c of inner edge 50 are certain to be positioned on connective necks 76 and not main body portion 74. Since the area of the main body portion 74 certain to be in capillary channel 25 is substantially greater than the area of connective necks 76, variation in the effective working electrode area in capillary channel 25 created by variations in the size and shape of inner edge 50 and by the placement of spacer substrate 20 on support substrate 12 is minimized.

Furthermore, measurement accuracy is improved by both connective necks 76 providing connectivity of working electrode 70 to contact 40 through at least lead 72. An electrode looping portion 78 extends under spacer substrate 20 from connective neck 76 on one side of working electrode 70 and is joined to lead 72 extending from the other connective neck 76 at a location adjacent to the capillary channel 25.

To the extent a biosensor comprising an electrode looping portion 78 is a desirable basic embodiment, the connective necks 76 further enable minimizing the effective area of the looping portion which minimizes the susceptibility of the electrode, particularly the working electrode, to electromagnetic interference.

FIG. 5 shows a portion of another embodiment of an electrode arrangement for biosensor 100, with features that can be employed in combination with any of the other features of the other biosensor embodiments discussed herein. Biosensor 100 includes capillary channel 25 with first counter electrode 60 and a second counter electrode 160. A working electrode 170 is positioned in capillary channel 25 between counter electrodes 60, 160. A sample sufficiency working electrode (SSWE) 180 is positioned at the end of capillary channel 25 opposite inlet 46 to detect when a sufficient volume of analyte sample is received in capillary channel 25. Working electrode 170 is similar to working electrode 70, and includes a main body portion 174 having length between opposite ends and a minimum width W1 located entirely in capillary channel 25. Connective necks 176 extend from the opposite ends of main body portion 174 and across inner edge 50. Connective necks 176 have a maximum width W2 that is substantially less than minimum width W1. Since the area of the main body portion 174 in certain to be located in capillary channel 25 is substantially greater than the area of connective necks 176 that varies in capillary channel 25, variation in the effective working electrode area in capillary channel 25 created by variations in the size of the channel formed by inner edge 50 and the placement of spacer substrate 20 on support substrate 12 is minimized. Furthermore, only one of connective necks 176 provides connectivity of working electrode 170 to contact 40. The other connective neck 176 extends to a sense lead connection 178, which extends along support substrate 12 to another contact (not shown) of biosensor 100.

FIG. 6 shows a portion of another embodiment of an electrode arrangement for biosensor 200, with features that can be employed in combination with any of the other features of the other biosensor embodiments discussed herein. Biosensor 200 includes capillary channel 25 with first counter electrode 60 and a second counter electrode 260. Counter electrodes 60, 260 extend across inner edge 50 to leads 62, 262 located along edge 32 of support substrate 12. A working electrode 270 is positioned in capillary channel 25 between counter electrodes 60, 260. A SSWE 280 and a sample sufficiency counter electrode (SSCE) 290 are positioned at the end of capillary channel 25 opposite inlet 46 to detect when a sufficient volume of analyte sample is received in channel 25. SSWE 280 and SSCE 290 extend along leads to contacts (not shown) on support substrate 12.

Working electrode 270 includes a main body portion with a pair of working electrode portions 274a, 274b spaced along its length. Working electrode portion 274a, 274b each have a minimum width W1 transverse to the length, and are sized to be located entirely in capillary channel 25. Necks 276 extend from the opposite ends of respective ones of the working electrode portions 274a, 274b and include sufficient lengths to extend across inner edge 50 to a location outside capillary channel 25. One of the necks 275 is a terminal neck, meaning generally that it terminates outside the capillary channel and does not extend or lead to another portion of the electrode 16, while the other neck 276 is connected with a lead that extends to at least one contact 40 on support substrate 12. Necks 276 each have a maximum width W2 that is substantially less than minimum width W1.

Furthermore, working electrode portions 274a, 274b are connected to one another by a connecting portion 278 having a maximum width W3 that is less than a minimum width of either of necks 276. Since the effective area of the working electrode portions 274a, 274b certain to be located in capillary channel 25 is substantially greater than the variation in the effective area of necks 276 caused by inner edge 50, variation in the effective working electrode area in capillary channel 25 is minimized.

FIG. 7 shows a portion of another embodiment electrode arrangement for biosensor 200′, which can be identical to biosensor 200 except as otherwise noted. Biosensor 200′ includes a capillary channel 25 with a working electrode 270′ positioned in capillary channel 25 between counter electrodes 60, 260. Working electrode 270′ includes a main body portion with a pair of working electrode portions 274a, 274b each having minimum width W1, and necks 276 extending from opposite ends of respective ones of the working electrode portions 274a, 274b and across inner edge 50. Necks 276 have maximum width W2 that is substantially less than minimum first width W1. Furthermore, main body portions 274a, 274b are connected to one another by a pair of connecting portions 278a, 278b that each has a maximum width W3 that is less than a minimum width of each of necks 276.

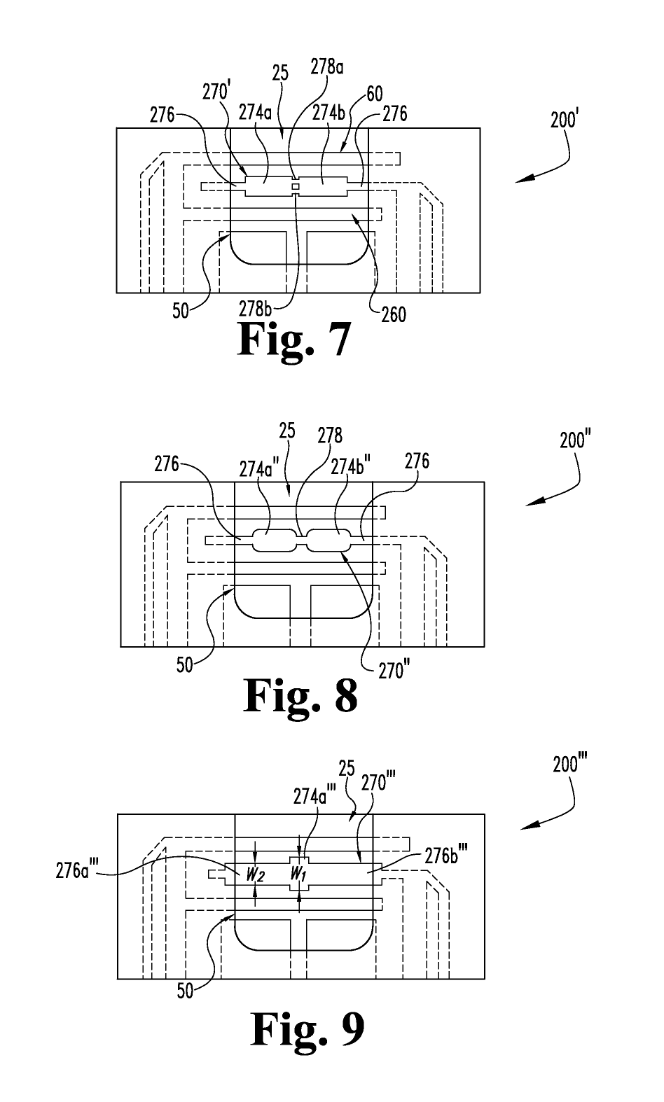

FIG. 8 shows a portion of another embodiment electrode arrangement for biosensor 200″, which can be identical to biosensor 200 except as otherwise discussed herein. Biosensor 200″ includes a capillary channel 25 with a working electrode 270″ positioned in capillary channel 25 between counter electrodes 60, 260. Working electrode 270″ includes a main body portion with a pair of working electrode portions 274a″, 274b″ each having a minimum width W1 located in capillary channel 25, and necks 276 extending from opposite ends of respective ones of working electrode portions 274a″, 274b″ and across inner edge 50. Necks 276 have a maximum width W2 that is substantially less than minimum width W1. Furthermore, main body portions 274a″, 274b″ are connected to one another by a connecting portion 278 that has a maximum width W3 that is less than the minimum width of connective necks 276.

Working electrode portions 274a″, 274b″ each include an oval shape that extends between the respective neck 276 and connecting portion 278. In one embodiment, the increased area of the working electrode portions is formed by adding electrode material to the location between neck 276 and connecting portion 278. In another embodiment, the increased area of the electrode portion is formed by removing or covering sufficient electrode material between and around main body portions 274a″, 274b″ to form connecting portion 278 and necks 276. For example, insulator material could be printed, or adhesive and/or spacer material placed, in capillary channel 25 to cover sufficient conductor material to form the desired shape and configuration of the main body portion.

FIG. 9 shows a portion of another embodiment electrode arrangement for biosensor 200′, which can be identical to the other biosensor embodiment 200 except as otherwise noted. Biosensor 200′″ includes a capillary channel 25 with a working electrode 270′″ positioned in capillary channel 25 between counter electrodes 60, 260. Working electrode 270′″ includes an outwardly projecting central body portion 274a′″ that has a minimum width W1 located in capillary channel 25, and lateral portions 276a′″, 276b′″ extending from opposite ends of the central body portion 274a′″ and across inner edge 50. Each of lateral portions 276a′″, 276b′″ has a maximum width W2 that is substantially less than first width W1. Furthermore, lateral portions 276a′″, 276b″ extend along a substantial portion of the length of working electrode 270′″ between opposite portions of edge 50 in capillary channel 25.

In one embodiment, lateral portions 276a″, 276b′ extend along at least 50% of the overall length of working electrode 270′″ between the opposite sides of inner edge 50. In another embodiment, lateral portions 276a″, 276b′ extend along at least 75% of the overall length of working electrode 270′″ between the opposite sides of inner edge 50. The outwardly projecting central body portion 274a′″ increases the effective area of the working electrode 270′ certain to be located in capillary channel 25, reducing the effect of variability in the effective working electrode area created by inner edge 50. Central body portion 274a′″ is formed in one embodiment by including additional conductor material to the working electrode between lateral portions 276a′″, 276b′″ to increase the width at or near the center of working electrode 270′″. Rather than include additional conductor material, the spacer may be configured (or insulative material added) so that the exposed width of lateral portions 276′″ is reduced, with the unreduced portion of the width forming central body portion 274a′″.

One useful aspect of certain of these embodiments is that the at least one connective portions of the embodiments of FIGS. 6-8 and the central body portion of the embodiment of FIG. 9 may be used as positive or negative registration patterns for purposes of manufacturing. For example, manufacturing equipment can be configured to optically detect the location of the connective portions or central body portion for determining proper placement of adhesive or of the spacer itself. In view of this disclosure, those of ordinary skill in the art will appreciate further useful aspects of these and other embodiments of the present invention.

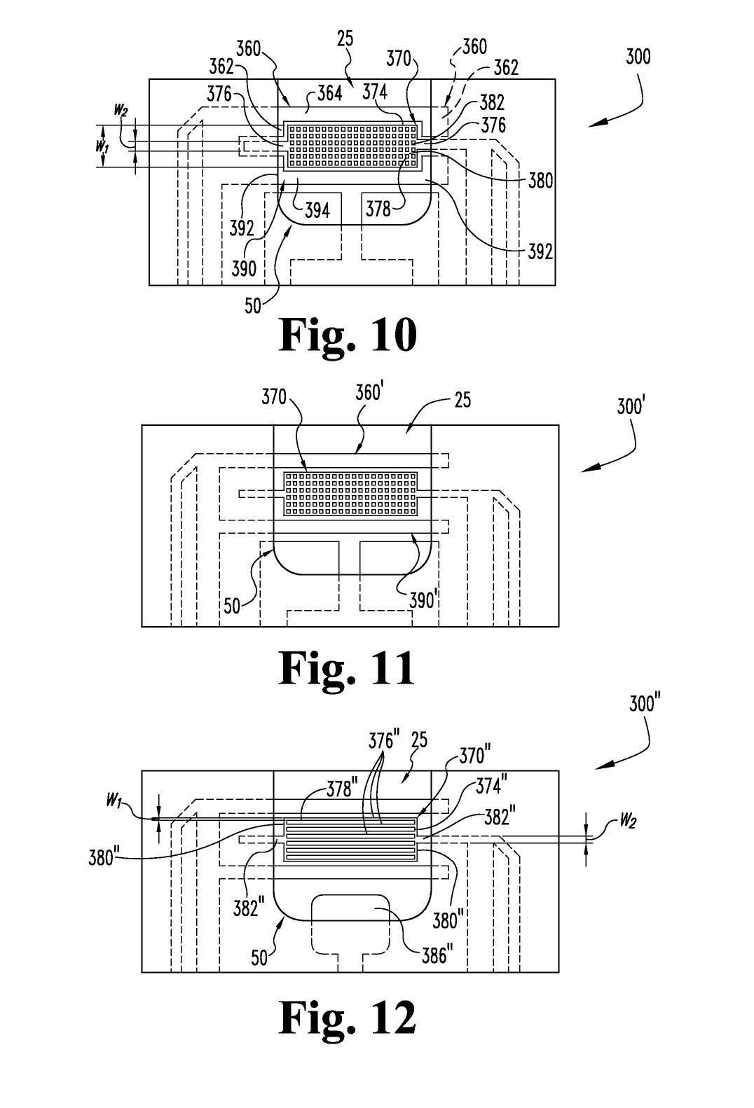

FIG. 10 shows another embodiment of biosensor 300 with features that can be employed in combination with any of the other features of the other biosensor embodiments discussed herein. Biosensor 300 includes a working electrode 370 with a main body portion 374 defining a minimum width W1 and opposite necks 376 extending from the ends of main body portion 374 and across inner edge 50 to a location outside capillary channel 25. Main body portion 374 is located within capillary channel 25. Necks 376 each define a maximum width W2 that is substantially less than minimum width W1. Main body portion 374 is comprised of a series of interconnected rows 378 and columns of working electrode portions 380 to faun a grid-shaped pattern. Non-conductive areas 382 lie between the rows and columns 378, 380. Each of the rows and columns 378, 380 defines a maximum width that is less than a minimum width of necks 376.

In the FIG. 10 embodiment, counter electrodes 360, 390 include thickened end portions 362, 392, respectively, that extend into capillary channel 25, and a central section 364, 394, respectively, that extend across capillary channel 25 to the respective end portions 362, 392. End portions 362, 392 and central sections 364, 394 frame the grid-shaped main body portion 374 of working electrode 370. Furthermore, inner edge 50 overlaps and extends along central section 394 and end portions 362, 392. The FIG. 11 embodiment is identical to biosensor 300, except that biosensor 300′ includes counter electrodes 360′, 390′ each including a uniform width extending entirely across capillary channel 25 and through inner edge 50 to a location outside capillary channel 25.

FIG. 12 shows another embodiment biosensor 300″ that is identical to the other biosensor embodiment 300′ except that it includes only an SSWE 386″ rather than a dual sample sufficiency electrode arrangement, and also includes another configuration of the working electrode 370. Working electrode 370″ includes a main body portion 374″ located within capillary channel 25. Main body portion 374″ is formed by a plurality of elongated rows 376″ of working electrode portions separated by respective ones insulated or non-conductive elongated row portions 378″. Main body portion 374″ also includes opposite working electrode end portions 380″ extending across the respective ends of rows 376″ to connect rows 376″ with respective ones of the necks 382″. Each of the rows 376″ defines a maximum width W1 and each of the necks 376″ defines a minimum width W2 that is greater than width W1. Furthermore, main body portion 374″ includes a minimum overall width at end portions 380″ that is greater than a maximum width of necks 382″.

One useful aspect of certain of these embodiments having the “open” areas or non-conductive portions of the working electrode that are completely or at least partially surrounded by conductive portions of the electrode, such as shown in FIGS. 10-12, is that the working electrode will behave like a planar electrode having an area corresponding to the actual area of the working electrode portions over short durations. Over longer durations, however, the working electrode will behave like a planar electrode having an area that encompasses both the actual area of the working electrode portions and the area of the bounded non-conductive portions. Thus, over time, the working electrode area appears to increase, allowing the biosensor to take advantage of the different time course of the current measured. The time constants for this change in current measurement are related to the diffusion coefficient of the electroactive substance in the measured fluid or sample substance. This allows information regarding the concentration and diffusion coefficient of the electroactive substance in the fluid to be obtained. The different time constants associated with the current measurements also allow separate measurement of capacitance and faradaic current since the capacitance is related to the actual conductive surface area of the working electrode, but at longer durations the faradaic current is related to the area of the working electrode surrounded or at least partially surrounded by the conductive working electrode portions, including the non-conductive portions. Thus, working electrodes can be made with a smaller ‘peak’ current, which aids in functioning of the current measurement device. In view of this disclosure, those of ordinary skill in the art will appreciate further useful aspects of these and other embodiments of the present invention.

FIG. 13 shows another embodiment biosensor 400 with features can be employed in combination with any of the other features of the other biosensor embodiments discussed herein. Biosensor 400 includes capillary channel 25 with a first counter electrode 460 and a second counter electrode 490. A working electrode 470 is positioned in capillary channel 25 between counter electrodes 460, 490. Working electrode 470 includes a main body portion 474 having a maximum first width W1 located in capillary channel 25, and necks 476 extending from opposite ends of main body portion 474 and across inner edge 50. Necks 476 each include a maximum width W2 that is substantially less than maximum width W1. Furthermore main body portion 474 tapers from maximum width W1 at or near the center of main body portion 474 to a minimum width W3 at the junction with respective ones of necks 476, where the minimum width W3 of main body portion 476 is greater than maximum width W2 of necks 476. Counter electrodes 460, 490 are arranged in an opposite manner so that each has a minimum width at or near its center that increases away from the minimum width toward the portions of inner edge 50 on opposite sides of counter electrodes 460, 490. This arrangement maximizes the working electrode area and counter electrode area in capillary channel 25 while also providing a greater effective area of working electrode area 470 certain to be located between the portions of inner edge 50 defining capillary channel 25 relative to the area of necks 476 certain to extend across inner edge 50.

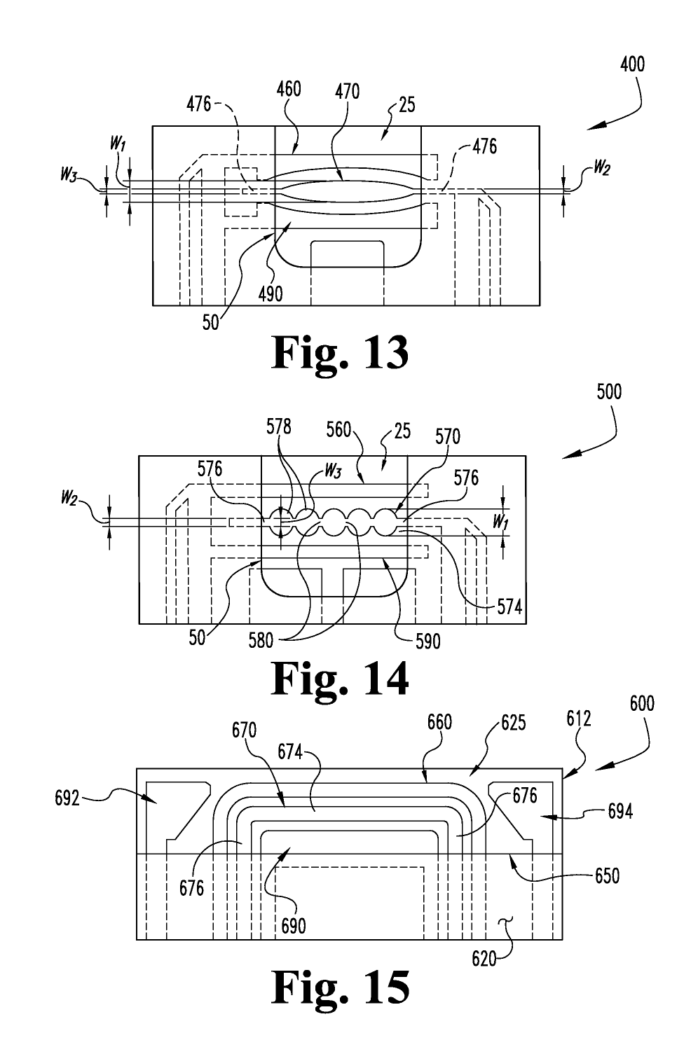

FIG. 14 shows another embodiment biosensor 500 with features that can be employed in combination with any of the other features of the other biosensor embodiments discussed herein. Biosensor 500 includes capillary channel 25 with a first counter electrode 560 and a second counter electrode 590. A working electrode 570 is positioned in capillary channel 25 between counter electrodes 560, 590. Working electrode 570 includes a main body portion 574 with a plurality of node shaped working electrode portions 578 connected to one another with connecting portions 580. Necks 576 extend from opposite sides of main body portion 574 and across inner edge 50. Working electrode portions 578 each have a maximum width W1 located in capillary channel 25, and necks 576 each have a maximum width W2 that is substantially less than first width W1. Connective portions 578 each include a maximum width W3 that is less than a minimum width of necks 576.

In the illustrated embodiment, working electrode portions 578 each include a substantially circular shape. Other embodiments contemplate other node-like shapes for working electrode portions 578, including oval, square, rectangular, polygonal, and non-circular shapes, for example. In the illustrated embodiment, the plurality of nodes include five node-shaped working electrode portions and the connecting portion includes four connecting portions, and adjacent pairs of the working electrode portions are connected by respective ones of the four connecting portions. Other embodiments contemplate two or more node-shaped portions with an appropriate number of connecting portions connecting the node-shaped portions.

FIG. 15 shows another embodiment biosensor 600 that is a fill width end dose biosensor. Biosensor 600 includes a capillary channel 625 that extends across the entire width of support substrate 612. The edge 650 of capillary channel is formed by spacer substrate 620. A first counter electrode 660 and a second counter electrode 690 extend across capillary channel 625, and a working electrode 670 is located in capillary channel 625 between counter electrodes 660, 690. SSCE 692 and SSWE 694 are located in capillary channel 625 adjacent the edges of support substrate 612. The portions of biosensor 600 not described can include any of the features of the biosensor embodiments discussed herein.

Working electrode 660 includes a main body portion 674 extending laterally between toward the side edges of support substrate 612, and opposite connective necks 676 that extend transversely from main body portion 674 toward the end of biosensor 10 opposite capillary channel 625. Spacer substrate 612 is positioned so that inner edge 650 extends across connective necks 676 and so that main body portion 674 is located entirely within capillary channel 625. This arrangement maximizes the area of working electrode 670 certain to be located in capillary channel 625 relative to the variation in effective working electrode area that may result due to the placement location of inner edge 650 along connective necks 676 and/or due to any irregularities in the boundaries of capillary channel 625 formed by inner edge 650.

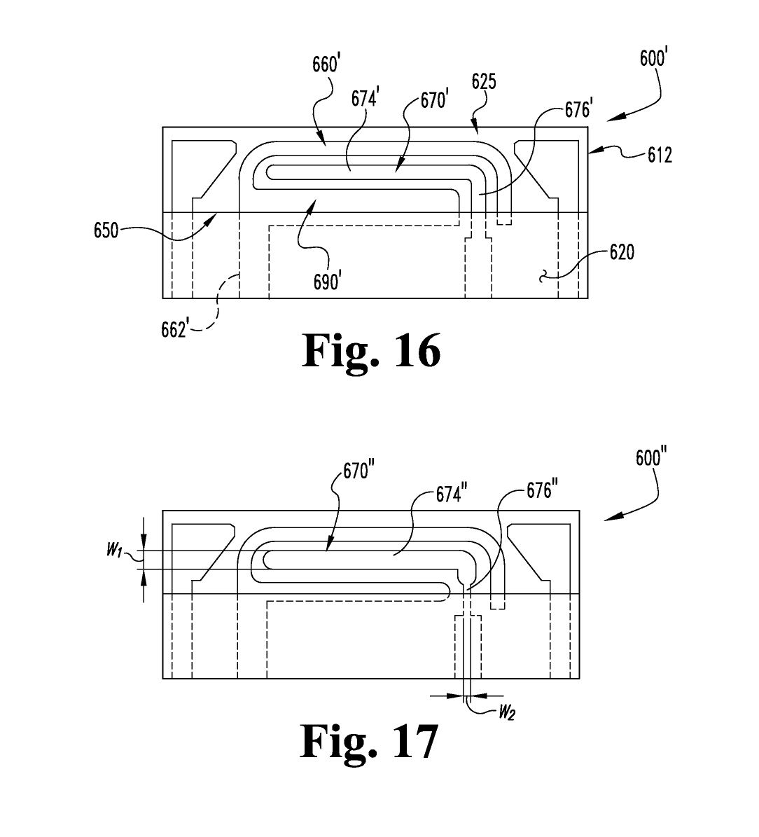

In FIG. 16 shows another embodiment of the biosensor 600 of FIG. 15. Biosensor 600′ includes a working electrode 670′ that includes only one connective neck 676′ extending from main body portion 674′ across inner edge 650. Thus, the effective area of the working electrode 670′ in capillary channel 625 formed by connective neck 676′ is half of that formed by the connective necks 676 of the FIG. 15 embodiment. Therefore, the area of main body portion 674′ of working electrode 670′ certain to be located in capillary channel 625 is substantially greater than any variation in effective working electrode area that may result due to the placement location of inner edge 650 along connective neck 676′ and/or due to any irregularities in the boundaries of capillary channel 625 formed by inner edge 650.

The embodiment of FIG. 16 further varies from the embodiment of FIG. 15 in the arrangement of counter electrodes. Counter electrodes 660′, 670′ are connected to a single lead 662′ along one side of support substrate 612. In both the FIG. 15 and FIG. 16 embodiments, inner edge 650 extends along and partially overlaps counter electrode 690, 690′.

In FIG. 17 another embodiment of the full width end dose biosensor 600′ of FIG. 16 is shown. Biosensor 600″ includes a working electrode 670″ that includes only one connective neck 676″ extending from main body portion 674″ across inner edge 650. Furthermore, working electrode 670″ includes a minimum width W1 along all or a substantial portion of its length that is substantially greater than a maximum width W2 of the portion of connective neck 676″ extending across inner edge 650. Thus, the area of working electrode 670″ certain to be located in capillary channel 625 is maximized and substantially greater than any variation in effective working electrode area that may result due to the placement location of inner edge 650 along the reduced width portion of connective neck 676″ and/or due to any irregularities in the boundaries of capillary channel 625 formed by inner edge 650.

In use, a number of the biosensors are typically packaged in a vial, usually with a stopper or other arrangement formed to seal the vial. It is appreciated, however, that the biosensors may be packaged individually, or biosensors can be folded upon one another, rolled in a coil, stacked in a cassette magazine, packed in blister packaging. In another embodiment, the packaging is formed as a card with removable individual segments comprised of biosensors, examples of which may be found in U.S. application Ser. No. 12/198,197 entitled “BIOSENSOR TEST STRIP CARDS,” the contents of which are incorporated herein by reference in its entirety. Since the biosensors include the herein described arrangements to maximize the area of the working electrode certain to be located in the capillary channel relative to the area of the portion of the working electrode affected by placement of the inner edge of the spacer substrate, the precision of the analyte measurements taken with the biosensors is improved.

Many fluid sample types may be analyzed using the biosensors discussed herein. For example, human body fluids such as whole blood, plasma, sera, lymph, bile, urine, semen, cerebrospinal fluid, spinal fluid, lacrimal fluid and stool specimens as well as other biological fluids readily apparent to one skilled in the art may be measured. Fluid preparations of tissues can also be assayed, along with foods, fermentation products and environmental substances, which potentially contain environmental contaminants. Preferably, whole blood is assayed with the biosensor.

A user of the biosensor places a finger having a blood collection incision or puncture against the inlet to the capillary channel. Capillary forces pull a liquid blood sample flowing from the incision or puncture into and through the capillary channel across the reagents and the electrodes in the capillary channel. The liquid blood sample dissolves the reagents and engages the electrodes in the capillary channel where the electrochemical reaction takes place.

Sometime after the reaction has begun, a power source (e.g., a battery) applies a potential difference between the electrodes respectively. When the potential difference is applied, the amount of oxidized form of the mediator at the reference or counter electrode and the potential difference must be sufficient to cause electro-oxidation of the reduced form of the mediator at the surface of the working electrode. A current measuring meter (not shown) measures the current generated by the oxidation of the reduced form of the mediator at the surface of the working electrode. The biosensors discussed herein minimize the variation in the working electrode area in the capillary channel, improving the accuracy and precision of the measured current from one biosensor to the next.

An example of a biosensor configured for use with electrochemical techniques is the ACCU-CHEK® Aviva test strip, which is described more fully in U.S. Patent Application Publication No. 2005/0016844, the disclosure of which is hereby incorporated herein by reference in its entirety. This exemplary test element is distributed in the United States by Roche Diagnostics Corporation of Indianapolis, Ind.

One illustrative method for manufacturing a biosensor includes providing a support substrate; forming at least one working electrode on the support substrate, the working electrode including a main body portion and at least one connective neck extending from an end of the main body portion, wherein a width of the at least one connective neck is greater than a minimum width of part of the main body portion of the working electrode; and positioning a spacer substrate on the support substrate, the spacer substrate including an inner edge that defines a boundary of a capillary channel, the inner edge extending across the at least one connective neck of the working electrode so that the part of the main body portion defining the minimum width is located entirely within the capillary channel.

In one refinement, the method may also include positioning a cover on at least the spacer substrate to form the capillary channel between the support substrate and the cover. In a further refinement of the method, the biosensor is a glucose sensor.

In another refinement of the method, the main body portion of the working electrode includes first and second working electrode portions and a connecting portion extending between the first and second working electrode portions, the connecting portion defining the part of the main body portion and first and second working electrode portions each define a minimum width in the capillary channel that is greater than the maximum width of the at least one connective neck. In a further refinement of the method, the connecting portion includes a plurality of connecting portions forming rows extending between the first and second working electrode portions, each of the connecting portions defining a width that corresponds to the minimum width. In yet a further refinement of the method, the first and second working electrode portions include a plurality of working electrode portions spaced along the plurality of connecting portions to form a grid-like pattern for the main body portion of the working electrode.

Another illustrative method for manufacturing a biosensor includes providing a support substrate; forming at least one working electrode on the support substrate, the working electrode including a main body portion defining a substantially constant width along a substantial portion of a length of the main body portion, the working electrode including a central portion projecting outwardly from the width; and positioning a spacer substrate on the support substrate so that opposite portions of an inner edge of the spacer substrate extend across opposite lateral portions of the main body portion and the central portion of the working electrode is positioned entirely within a capillary channel defined by portions of the inner edge, wherein the central portion occupies less than half of the length of the main body portion between the portions of the inner edge. In a refinement of the method, the central portion occupies less than one fourth of the length of the main body portion between the portions of the inner edge.

Further details and examples of conventional blood glucose meters and related electrical and optical components and their respective measurement techniques are described in U.S. Pat. Nos. 5,352,351; 4,999,482; 5,438,271; 6,645,368; 5,997,817; 6,662,439; RE 36,268; 5,463,467; 5,424,035; 6,055,060; 6,906,802; and 5,889,585; the disclosures of which are hereby incorporated herein by reference in their entireties.

While the invention has been illustrated and described in detail in the drawings and foregoing description, the same is to be considered as illustrative and not restrictive in character, it being understood that only certain embodiments have been shown and described and that all changes and modifications that come within the spirit of the inventions are desired to be protected. It should be understood that while the use of words such as preferable, preferably, preferred or more preferred utilized in the description above indicate that the feature so described may be more desirable, it nonetheless may not be necessary and embodiments lacking the same may be contemplated as within the scope of the invention, the scope being defined by the claims that follow. In reading the claims, it is intended that when words such as “a,” “an,” “at least one,” or “at least one portion” are used there is no intention to limit the claim to only one item unless specifically stated to the contrary in the claim. When the language “at least a portion” and/or “a portion” is used the item can include a portion and/or the entire item unless specifically stated to the contrary.

What is claimed is:

1. A biosensor, comprising:
a support substrate extending between opposite first and second ends and opposite first and second edges;
a spacer substrate positioned on the support substrate, the spacer substrate including an inner edge extending along the support substrate between one or both of the first and second ends and the first and second edges;
a cover cooperating with the spacer substrate, the inner edge of the spacer substrate defining a boundary of a capillary channel; and
at least one working electrode in the capillary channel, and the working electrode including a width and a main body portion extending along a length transversely to the width between opposite ends of the main body portion, the main body portion including at least two working electrode portions positioned along the length of the main body portion in the capillary channel with the at least two working electrode portions connected by at least one connecting portion, the working electrode further including at least one connective neck extending from at least one of the opposite ends of the main body portion and across the inner edge of the spacer substrate, wherein the two working electrode portions each define a minimum width that is greater than a maximum width of the connective neck, and the connecting portion defines a maximum width that is less than a minimum width of the connective neck.

2. The biosensor of claim 1, wherein the capillary channel includes an inlet at the first end of the support substrate and the main body portion of the working electrode is located entirely within the capillary channel.

3. The biosensor of claim 1, wherein the working electrode includes a second neck extending from the other of the opposite ends of the main body portion in the capillary channel, the second neck extending across the inner edge of the spacer substrate.

4. The biosensor of claim 1, further comprising at least one counter electrode in the capillary channel and each of the at least one counter electrode is positioned substantially adjacent the working electrode.

5. The biosensor of claim 1, wherein the working electrode includes first and second connecting portions extending between and connecting the at least two working electrode portions to one another in the capillary channel, wherein the first and second connecting portions each include a maximum width that is less than the minimum width of the connective neck, and the first and second connecting portions are separated from one another by a non-conductive space between the connecting portions and the working electrode portions.

6. The biosensor of claim 1, wherein each of the working electrode portions includes one of a rectangular shape, a circular shape and an oval shape.

7. The biosensor of claim 1, wherein the at least two working electrode portions include five working electrode portions and the at least one connecting portion includes four connecting portions, and adjacent pairs of the working electrode portions are connected by respective ones of the four connecting portions.

8. The biosensor of claim 1, wherein the at least one connecting portion of the working electrode includes a plurality of rows of connecting portions extending between the at least two working electrode portions of the working electrode, and adjacent pairs of the rows of connecting portions are separated from one another by a non-conductive space, each of the rows of connecting portions including a maximum width that is less than the minimum width of the at least one connective neck.

9. The biosensor of claim 8, wherein the at least two working electrode portions of the main body portion of the working electrode includes a plurality of working electrode portions spaced along the plurality of rows of connecting portions to form a grid-shaped pattern for the main body portion of the working electrode.

10. A biosensor, comprising:
a support substrate extending between opposite first and second ends and opposite first and second edges;
a spacer substrate positioned on the support substrate, the spacer substrate including an inner edge extending along the support substrate, the inner edge being located between the first and second ends and the first and second edges of the support substrate;
a cover cooperating with the spacer substrate, the inner edge of the spacer substrate defining a boundary of a capillary channel; and
at least one working electrode that includes a main body portion defining a width and a length transverse to the width between opposite ends of the main body portion, the length and width being sized so that the main body portion is located in the capillary channel, the working electrode further including first and second connective necks that each extend from a respective one of the opposite ends of the main body portion and across the inner edge of the spacer substrate, the main body portion defining a minimum width that is greater than a maximum width of each of the first and second necks, wherein each of the first and second connective necks extends from the main body portion to an electrode lead on the support substrate so that each of the first and second connective necks provides an electrical connection between the working electrode and at least one contact configured to connect the biosensor to a meter.

11. The biosensor of claim 10, wherein the opposite first and second ends and the opposite first and second edges of the support substrate form a rectangular shape.

12. The biosensor of claim 10, wherein the capillary channel is located at the first end of the support substrate and the capillary channel includes an inlet at the first end between the support substrate and the cover.

13. The biosensor of claim 12, wherein the inner edge defines a generally U-shaped configuration and the main body portion of the working electrode is located entirely within the capillary channel.

14. The biosensor of claim 10, wherein the main body portion of the working electrode includes a maximum width at or near a center of the main body portion and tapers in width from the maximum width to the minimum width adjacent each of the first and second connective necks.

15. The biosensor of claim 10, wherein the first connective neck extends to an electrode lead that extends along the support substrate to the at least one contact, and the second connective neck extends to an electrode looping portion located outside the capillary channel, the electrode looping portion joining the second connective neck to the electrode lead so that the working electrode forms a continuous loop located within and outside the capillary channel.

16. A biosensor, comprising:
a support substrate extending between opposite first and second ends and opposite first and second edges;
a spacer substrate positioned on the support substrate, the spacer substrate including an inner edge extending along the support substrate, the inner edge extending from the first edge to the second edge adjacent the first end of the support substrate;
a cover cooperating with the spacer substrate, the inner edge of the spacer substrate defining a boundary of a capillary channel; and
at least one working electrode in the capillary channel, and the working electrode includes a main body portion within the capillary channel having a length that extends toward the first and second edges, the working electrode further including a connective neck extending from an end of and transversely to the main body portion toward the second end of the support substrate, wherein the inner edge is spaced from the main body portion and extends across the connective neck where the connective neck is oriented toward the second end of the support substrate.

17. The biosensor of claim 16, wherein the main body portion of the working electrode is located entirely within the capillary channel.

18. The biosensor of claim 16, wherein the working electrode includes first and second connective necks extending from opposite ends of and transversely to the main body portion toward the second end of the support substrate and the inner edge extends across each of the first and second connective necks where the first and second connective necks are oriented toward the second end of the support substrate.

19. The biosensor of claim 16, wherein the connective neck provides the sole electrical connection of the main body portion extending across the inner edge.

20. The biosensor of claim 16, wherein the main body portion includes a minimum width along a substantial portion of the length and the connective neck includes a maximum width in a direction extending between the first and second edges of the support substrate, the minimum width of the main body portion being greater than the maximum width of the connective neck.

21. The biosensor of claim 16, further comprising first and second counter electrodes in the capillary channel, wherein:
the main body portion of the working electrode is located between the first and second counter electrodes;
the first counter electrode is located between the first end of the support substrate and the working electrode; and
the second counter electrode includes a body portion oriented between the first and second edges and the inner edge extends along the body portion of the second counter electrode.
